(19) United States
(12) Patent Application Publication
(10) Pub. No.: US 2024/0103006 A1
(43) Pub. Date: Mar. 28, 2024

(54) HIGHLY WATER-SOLUBLE AND STABLE CHEMOSENSOR FOR CYSTEINE

(71) Applicant: Roche Diagnostics Operations, Inc., Indianapolis, IN (US)

(72) Inventors: Maksim Fomin, Bergisch Gladbach (DE); Hannes Kuchelmeister, München (DE)

(21) Appl. No.: 18/468,249

(22) Filed: Sep. 15, 2023

Related U.S. Application Data
(63) Continuation of application No. PCT/EP2022/056065, filed on Mar. 9, 2022.

(30) Foreign Application Priority Data
Mar. 15, 2021 (EP) 21162688.2

(51) Int. Cl. G01N 33/68; C07C 259/06

(52) U.S. Cl. CPC G01N 33/6845; C07C 259/06; G01N 33/6842

(57) ABSTRACT

The present invention relates to chemical probes for the improved detection of cysteine in a test sample, preferably an aqueous test sample, as well as respective uses and kits.

### CROSS-REFERENCE TO RELATED APPLICATIONS

This application is a continuation of International PCT Application No. PCT/EP2022/056065 filed on Mar. 9, 2022, which claims priority to European Patent Application No. 21162688.2 filed on Mar. 15, 2021, the contents of each application are incorporated herein by reference in their entireties.

The present invention relates to chemical probes for the improved detection of cysteine in a test sample, preferably an aqueous test sample, as well as respective uses and kits.

### BACKGROUND OF THE INVENTION

Cysteine (Cys) is important in biosynthesis, detoxification, and metabolism. An elevated level of total cysteine can predict cardiovascular disease and metabolic syndromes. Cysteine deficiency is known to be one of the consequences of aging. The selective detection of Cys over structurally similar homocysteine (Hcy) or glutathione (GSH) remains an immense challenge. Although there are many methods for detecting Cys, photoluminescence (PL) and electrochemiluminescence (ECL) techniques are well-suited for clinical diagnostics and analytical technology because of their high sensitivities.

Trisulfide formation in recombinant monoclonal antibodies is a source of heterogeneity which needs to be controlled for consistent product quality. Ryll et al. (Kshirsagar, R.; McElearney, K.; Gilbert, A.; Sinacore, M.; Ryll, T. Biotechnol. Bioeng. 2012, 109, 2523) have shown that the L-cysteine (Cys) concentration in the feed medium directly correlated with the trisulfide level in the product (IgG1 mAb). Therefore, controlling of Cys feed strategies is required to lower trisulfide formation to acceptable levels.

Up to now, the detection of Cys attracts a lot of attention for various biochemical applications. A number of methods for detecting Cys have been developed such as fluorometry, potentiometry, electrochemical voltammetry and HPLC combined with Ellman's reagent or coupled with fluorescence. These methods require complicated instrumentation, involve cumbersome laboratory procedures or are low throughput.

Kim and Hong (in: Photoluminescence and Electrochemiluminescence Dual-Signaling Sensors for Selective Detection of Cysteine Based on Iridium(III) Complexes. ACS Omega 2019, 4, 7, 12616-12625) report PL and ECL dual-channel sensors using cyclometalated iridium(III) complexes for the discrimination of Cys from Hcy and GSH.

UV-vis spectrometry provides fast and simple measurement procedures. Thus, to quantify essential metabolites in bioprocesses, photometric assays are employed using automated analyzers, such as Cedex Bio HT (Roche Diagnostics, Penzberg, Germany). But only few candidates can be utilized for colorimetric Cys detection on Cedex Bio HT Analyzer (“Cedex”), and the analytical device offers only a limited set of wavelengths: 340, 378, 409, 480, 512, 520, 552, 583, 629, 652, 659, and 800 nm. In addition, an ideal probe for Cedex system must comprise high sensitivity, rapid response, aqueous solubility and stability, and ease of use.

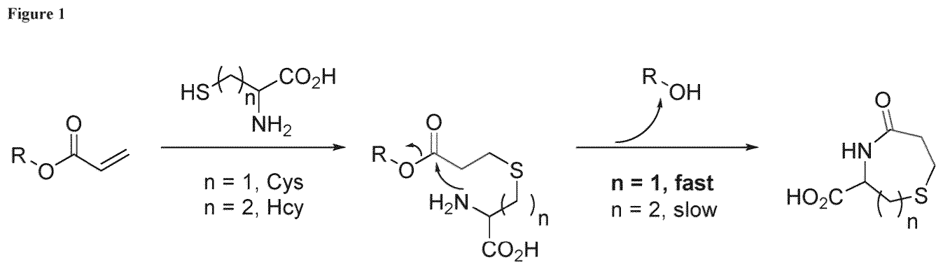

To date, most of the indicators for Cys are based on the strong nucleophilicity of the thiol group. Various mechanisms have been employed, including Michael addition and cleavage reactions. A sensing strategy based on acrylate group seemed promising, because it allowed to discriminate Cys from other amino acids and thiols (Han, Q.; Shi, Z.; Tang, X.; Yang, L.; Mou, Z.; Li, J.; Shi, J.; Chen, C.; Liu, W.; Yang, H.; Liu, W. Organic & Biomolecular Chemistry 2014, 12, 5023). The sensing mechanism is given in FIG. 1. This strategy involves the conjugate addition of Cys to acrylate to generate thioesters followed by an intramolecular cyclization. The acrylate moiety as thiol activation site undergoes fast cyclization only with Cys, since the reaction rate depends strongly on the ring size of the resulting lactam. After the masking acrylate group is removed, the conjugated π-electron system of the chromophore is restored, which enables the colorimetric response.

Disadvantageously, only few acrylate-based probes feature an intense colorimetric response at the wavelength required for the use on Cedex. Their chromophores are based on xanthene, merocyanine, heptamethine and fluorescein. The fact that most of reported acrylates were applied in organic solvent-water mixtures is obviously necessitated by their poor solubility in aqueous media. However, it is a must for applications on Cedex that probes are soluble in water, since parts of the instrument are labile to organic solvents. Also, the stability of existing probes has to be evaluated in order to ensure that an assay solution can be stored in Cedex for reasonable amount of time.

In view of these and other disadvantages, it is an object of the present invention to provide Cys probes that which can be used in aqueous solutions and provide a sufficient colorimetric response at one of the required wavelengths. Other objects and advantages will become apparent to the person of skill when studying the present description of the present invention.

In a first aspect of the present invention, the above object is solved by a compound according to Formula (I),

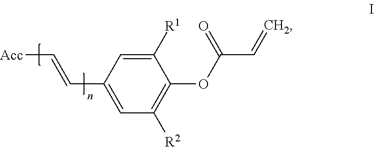

wherein
R¹ and R² are independently selected from R³, O—R³, S—R³, SO₃⁻, SO₃—R₃, wherein R³ is selected from C₁-C₁₈ alkyl, and a polyethylene glycol (PEG) residue,
Acc is selected from the group selected from Formula II

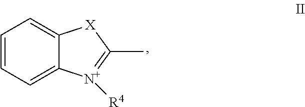

wherein X is selected from —N(CH₃)—, —S—, —Se—, —O—, and —C(CH₃)₂—,

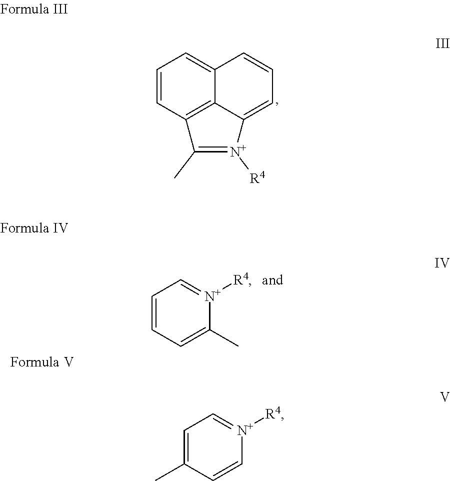

wherein optionally in each of Formula II to V an aromatic ring is substituted with 1, 2 or 3 SO₃⁻ groups,
R⁴ is selected from the group of C₁-C₁₈ alkyl, C₁-C₆ cycloalkyl, and (CH₂)ₘ—SO₃⁻, wherein m is an integer selected from 1 to 18, and n is selected from 1, 2 and 3, and suitable salts and solvates thereof.

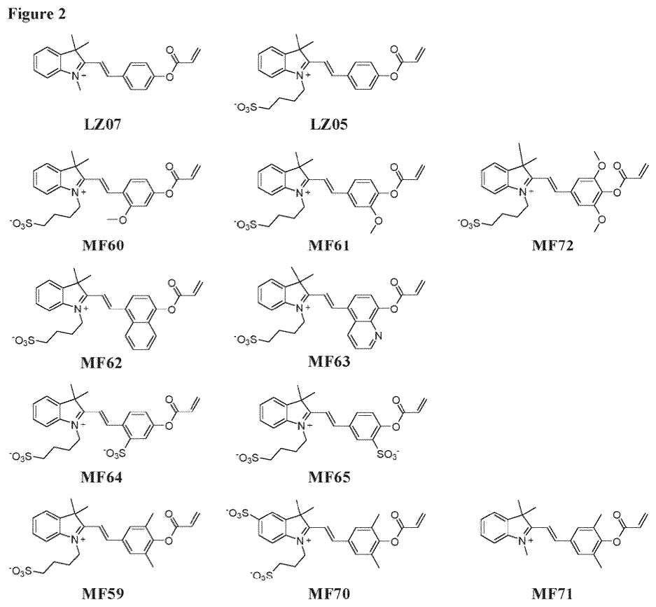

The present inventors synthesized a series of acryloyl esters based on merocyanine chromophore (FIG. 2, see below); probe LZ07 was prepared as a control and for comparison, and is known from the literature (Han, Q.; Shi, Z.; Tang, X.; Yang, L.; Mou, Z.; Li, J.; Shi, J.; Chen, C.; Liu, W.; Yang, H.; Liu, W. Organic & Biomolecular Chemistry 2014, 12, 5023). The present inventors then studied the spectral properties, aqueous solubility and stability of the acryloyl esters, and evaluated the response to Cys. It could be demonstrated that the chemical design provided dedicated probes, in particular for Cedex.

Preferred is the compound of Formula I according to the present invention, wherein R¹ and R² are independently selected from R³, O—R³, S—R³, SO₃⁻, SO₃—R³, wherein R³ is selected from C₁-C₆ alkyl, and a polyethylene glycol (PEG) residue,
Acc is Formula II

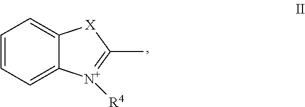

wherein X is selected from —N(CH₃)—, —S—, —Se—, —O—, and —C(CH₃)₂—, optionally the aromatic ring is substituted with 1, 2 or 3 SO₃⁻ groups, R⁴ is selected from C₁-C₆ alkyl, C₁-C₆ cycloalkyl, and (CH₂)ₘ—SO₃⁻, wherein m is an integer from 1 to 6, and n is 1, and suitable salts and solvates thereof.

Further preferred is the compound of Formula I according to the present invention, wherein R¹ and R² are independently selected from R³, O—R³, S—R³, SO₃⁻, SO₃—R³, wherein R³ is selected from C₁-C₃ alkyl, and a polyethylene glycol (PEG) residue,
Acc is Formula II

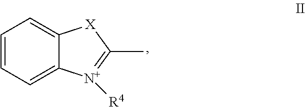

wherein X is —C(CH₃)₂—, optionally the aromatic ring is substituted with 1, 2 or 3 SO₃⁻ groups, R₄ is (CH₂)ₘ—SO₃⁻, wherein m is an integer from 1 to 6, and n is 1, and suitable salts and solvates thereof.

Further preferred is the compound of Formula I according to the present invention according to the following formulae VI to IX

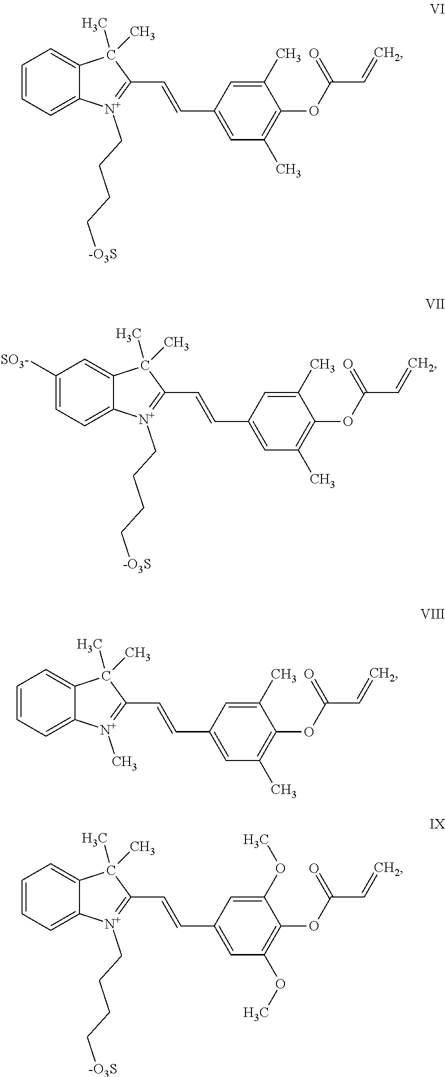

and suitable salts and solvates thereof.

In the context of the present invention, a suitable salt is usually one that does not interfere or does not substantially interfere with the solubility of the compound according to the present invention, in particular with the solubility in aqueous media. Examples are salts containing Group I elements (Li⁺, Na⁺, K⁺, Cs⁺, Rb⁺), the ammonium ion (NH₄⁺), the nitrate ion (NO₃⁻), containing Cl⁻, Br⁻, or I⁻, or sulfate salts.

Yet another aspect of the present invention then relates to a method for preparing a compound according to Formula I according to the present invention, comprising the steps of:
a) suitably reacting a compound of Formula VI

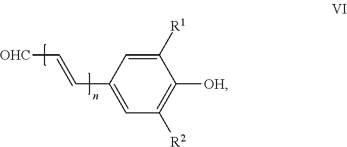

wherein R¹ and R² are as defined as above, and n is 1 or 2, with a compound of Formula II

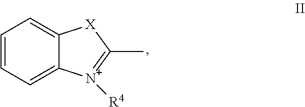

or with a compound of formula III

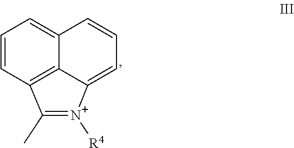

or with a compound of Formula IV

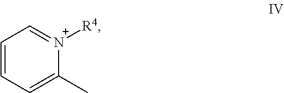

or with a compound of Formula V

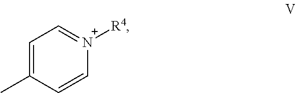

wherein in each of Formulae II to V optionally an aromatic ring is substituted with 1, 2 or 3 SO₃⁻ groups, R⁴ is selected from C₁-C₁₈ alkyl, C₁-C₆ cycloalkyl, and (CH₂)ₘ—SO₃⁻, and wherein m is an integer from 1 to 18, to obtain a compound of Formula VIII

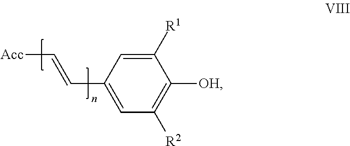

wherein R¹, R² and Acc are as defined as above, and n is 1 or 2, and b) suitably reacting the compound of formula VIII with acryloyl chloride. Suitable conditions for performing the above method are known to the person of skill in the art, and are exemplary disclosed in the examples and schemes, below.

Yet another aspect of the present invention then relates to a method for detecting cysteine in a test sample, comprising the following steps of: a) Measuring of UV/Vis absorbance of a solution of a compound as defined according to the present invention in a suitable solvent before and after being contacted with a prospectively cysteine-containing test sample, and b) Determining the difference in absorbance by comparison of the UV/Vis spectra as measured in step a), and c) Detecting cysteine in said test sample based on said difference in absorbance as determined in step b).

Test samples according to the present invention can comprise any sample comprising or prospectively comprising cysteine. Examples are, for example, the detection of biothiols in plasma, in samples obtained from patients, in total proteins in different kinds of cell lines, in tissue samples, cell lysates, serum, saliva or urine, in antibody samples, and in samples used in biotechnological applications. Preferred are aqueous biological samples to be analyzed in a Cedex-system.

Spectra recorded in the presence of Cys confirmed a colorimetric response through the cleavage of acryloyl ester. Probes as synthesized showed significant bathochromic shifts to the green and yellow range of the visible spectrum (Table 2). Preferred is the method according to the present invention, wherein the UV/Vis absorbance is measured at discrete wavelengths in the range of from 200 nm to 1000 nm. Moreover, their spectral profiles were advantageously fulfilling the wavelength requirement for the Cys sensing application in Cedex system (340, 378, 409, 480, 512, 520, 552, 583, 629, 652, 659 and 800 nm).

More preferred is the method according to the present invention, wherein the solvent is an aqueous solvent.

In a preferred aspect of the present invention, the method according to the present invention, wherein said determining the difference in absorbance is by a visual inspection of a color change, such a significant bathochromic shift to the green and yellow range of the visible spectrum. As an example, in a slightly different approach, Hai-Feng Yin, et al. (in: simple probe with visible color change for selective detection of cysteine, Spectroscopy Letters, (2020) DOI: 10.1080/00387010.2020.1821063) synthesized a fluorescent probe, which can selectively detect cysteine. With addition of cysteine, the probe solution showed marked color change from pale yellow to orange color by the naked eye.

The method according to the present invention, wherein said determining the difference in absorbance is by Cedex Bio HT (Roche Diagnostics, Penzberg, Germany).

Yet another aspect of the present invention then relates to a kit for detecting cysteine in a test sample, comprising a vial or container comprising a predetermined quantity of a compound according to the present invention, together with a manual for using said kit. Examples of included materials are, for example, a standard, a probe according to the invention, and buffer(s).

Yet another aspect of the present invention then relates to the use of a compound according to the present invention, or a kit according to the present invention for detecting cysteine in a test sample, preferably an aqueous test sample as disclosed herein.

The present invention will now be described further in the following examples, and also with reference to the Figures, nevertheless, without being limited thereto. For the purposes of the present invention, all references as cited herein are incorporated by reference in their entireties.

FIG. 1 shows a scheme of the mechanism of reaction of acryloyl esters with Cys (R—OH=merocyanine).

FIG. 2 shows the structure of probes as synthesized in the context of the present invention.

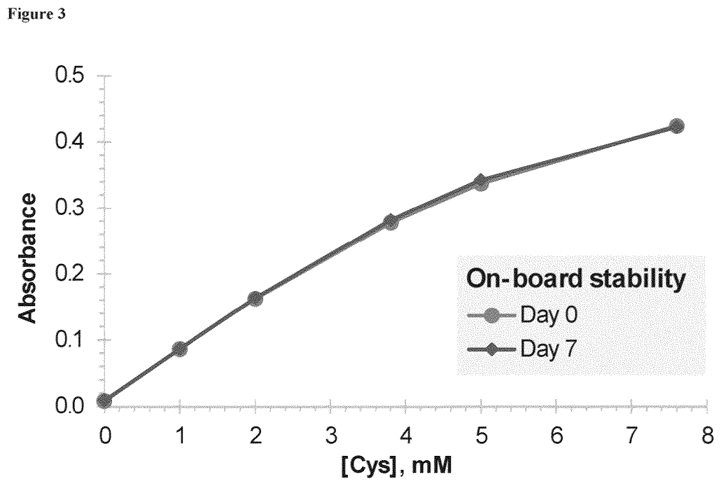

FIG. 3 shows the results of calibration in a feed medium (DMT118F.01 w/o Cys). SR: MF70 in DMSO/water (1:1). R1: 100 mM K—PO₄.

### EXAMPLES

A series of acryloyl esters based on merocyanine chromophore was synthesized (FIG. 2), and was compared with probe LZ07 as known from the literature (Han, Q.; Shi, Z.; Tang, X.; Yang, L.; Mou, Z.; Li, J.; Shi, J.; Chen, C.; Liu, W.; Yang, H.; Liu, W. Organic & Biomolecular Chemistry 2014, 12, 5023). Spectral properties, aqueous solubility and stability were studied, and the response to Cys was evaluated. The inventors demonstrated that the chemical design according to the present invention provides dedicated probes for Cedex.

The following is a brief summary of the state of the art regarding know Cys-probes and their properties:

| Probe | Solvent | Spectra | Reference |
|---|---|---|---|
| 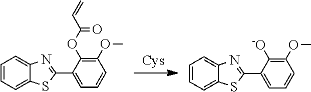 | Assay EtOH/H2O (2:8, v/v) solution buffered at pH 7.4 (phosphate buffer, 20 mm) | | Yang, X.; Guo, Y.; Strongin, R. M. Angew. Chem. Int. Ed. 2011, 50, 10690. |
| 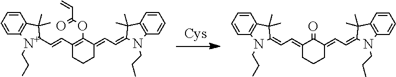 [SMILES: C=CC(=O)OC1=C(/C=C/C2=[N+](CCC)c3ccccc3C2(C)C)CCC/C1=C\C=C1\N(CCC)c2ccccc2C1(C)C ; CCCN1/C(=C/C=C2\CCC/C(=C\C=C3\N(CCC)c4ccccc4C3(C)C)C2=O)C(C)(C)c2ccccc21] | Assay EtOH : HEPES (1:9, pH 7.4, 0.01 M) | Probe 775 nm With Cys 515 nm | Guo, Z.; Nam, S.; Park, S.; Yoon, J. Chemical Science 2012, 3, 2760. |
| 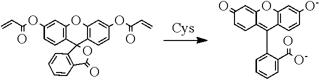 [SMILES: C=CC(=O)Oc1ccc2c(c1)Oc1cc(OC(=O)C=C)ccc1C21OC(=O)c2ccccc21 ; O=C([O-])c1ccccc1-c1c2ccc(=O)cc-2oc2cc([O-])ccc12] | Assay ethanol- phosphate buffer (20 mM, pH 7.4, 2:8 v/v) | With Cys 490 nm | Wang, H.; Zhou, G.; Gai, H.; Chen, X. Chem. Commun. 2012, 48, 8341. |
| 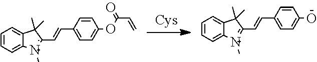 [SMILES: C=CC(=O)Oc1ccc(/C=C/C2=[N+](C)c3ccccc3C2(C)C)cc1 ; C[N+]1=C(/C=C/c2ccc(O)cc2)C(C)(C)c2ccccc21] | | Probe 384 nm With Cys 512 nm | Han, Q.; et al. Organic & Biomolecular Chemistry 2014, 12, 5023. |
| 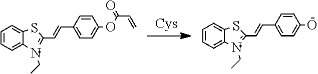 [SMILES: C=CC(=O)Oc1ccc(/C=C/c2sc3ccccc3[n+]2CC)cc1 ; CC[n+]1c(/C=C/c2ccc([O-])cc2)sc2ccccc21] | | | Li, H.; Jin, L.; Kan, Y.; Yin, B. Sensors and Actuators B: Chemical 2014, 196, 546. |
| 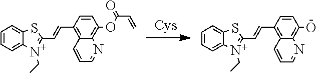 [SMILES: C=CC(=O)Oc1ccc(/C=C/c2sc3ccccc3[n+]2CC)c2cccnc12 ; CC[n+]1c(/C=C/c2ccc(O)c3ncccc23)sc2ccccc21] | | Probe 387 nm With Cys 566 nm | |
| 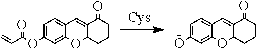 [SMILES: C=CC(=O)Oc1ccc2c(c1)OC1CCCC(=O)C1=C2 ; O=C1CCCC2Oc3cc(O)ccc3C=C12] | | Probe 302, 358 nm With Cys 398 nm | Lee, Y. H.; et al. Chem. Commun. 2015, 51, 14401. |
| 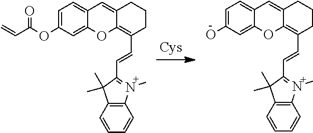 [SMILES: C=CC(=O)Oc1ccc2c(c1)OC1=C(/C=C/C3=[N+](C)c4ccccc4C3(C)C)CCCC1=C2 ; C[N+]1=C(/C=C/C2=C3Oc4cc([O-])ccc4C=C3CCC2)C(C)(C)c2ccccc21] | | Probe 582 nm With Cys 674 nm | Zhang, J.; et al. Anal. Chem. 2015, 87, 4856. |
| 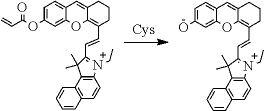 [SMILES: C=CC(=O)Oc1ccc2c(c1)OC1=C(/C=C/C3=[N+](CC)c4ccc5ccccc5c4C3(C)C)CCCC1=C2 ; CC[N+]1=C(/C=C/C2=C3Oc4cc([O-])ccc4C=C3CCC2)C(C)(C)c2c1ccc1ccccc21] | | Probe 608, 572 nm With Cys 710, 684, 632 nm | Han, C.; et al. ACS Applied Materials & Interfaces 2015, 7, 27968. |
| 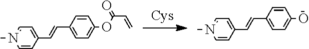 | | Probe 335 nm With Cys 372 nm | Niu, W.; et al. Anal. Chem. 2016, 88, 1908. |
| 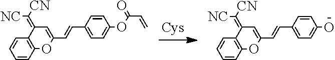 [SMILES: C=CC(=O)Oc1ccc(/C=C/C2=CC(=C(C#N)C#N)c3ccccc3O2)cc1 ; N#CC(C#N)=C1C=C(/C=C/c2ccc(O)cc2)Oc2ccccc21] | | Probe 381 nm With Cys 557 nm | Wang, J.; et al. ACS Sensors 2016. |
| 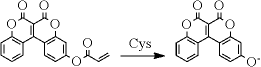 [SMILES: C=CC(=O)Oc1ccc2c(c1)oc(=O)c1c(=O)oc3ccccc3c12 ; O=c1oc2ccccc2c2c1c(=O)oc1cc([O-])ccc12] | Stock solution DMSO Assay DMSO- PBS | Probe 350 nm With Cys 445 nm | Chen, C.; Zhou, L.; Huang, X.; Liu, W. Journal of Materials Chemistry B 2017, 5, 5892. |
| 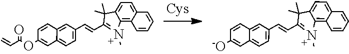 | PBS | With Cys 460 nm | Dai, X.; Kong, X.; Lin, W. Dyes and Pigments 2017, 142, 306. |
| 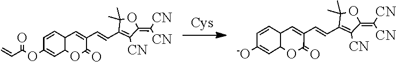 | Stock solution DMSO Assay PBS buffer solution (10 mM, | Probe 423 nm With Cys 335, 584 nm | Feng, S.; et al. Dyes and Pigments 2017, 146, 103. |
| | pH 7.4, with 10% | | |
| | DMSO, v/v) | | |
| 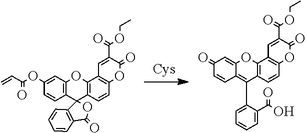 [SMILES: C=CC(=O)Oc1ccc2c(c1)Oc1c(ccc3oc(=O)c(C(=O)OCC)cc13)C21OC(=O)c2ccccc21 ; CCOC(=O)c1cc2c(ccc3c(-c4ccccc4C(=O)O)c4ccc(=O)cc-4oc32)oc1=O] | Assay DMSO-PBS buffer solution (10.0 mM, pH = 7.4, 3:7 (v/v)) | Probe 332 nm With Cys 450 nm, | Fu, Z .-H.; et al. Anal. Chem. 2017, 89, 1937. |
| 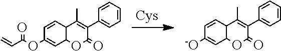 | Assay PBS containing 10% CH3CN | Probe 315 nm With Cys 380 nm | Kang, Y.-F.; et al. Aust. J. Chem. 2017, 70, 952. |
| 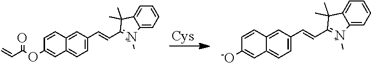 | Assay PBS containing 1% DMSO | Probe 409 nm With Cys 448 nm | Liu, G.; et al. J. Talanta 2017, 170, 406. |
| 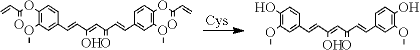 [SMILES: C=CC(=O)Oc1ccc(/C=C/C(=O)/C=C(O)/C=C/c2ccc(OC(=O)C=C)c(OC)c2)cc1OC ; COc1cc(/C=C/C(=O)/C=C(O)/C=C/c2ccc(O)c(OC)c2)ccc1O] | Assay phosphate buffer C2H5OH (pH 7.4, v/v, 1:1) | Probe 400 nm With Cys 427 nm. | Pang, L.; et al. Industrial & Engineering Chemistry Research 2017, 56, |
| | | | 7650. |
| 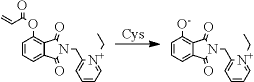 [SMILES: C=CC(=O)Oc1cccc2c1C(=O)N(Cc1cccc[n+]1CC)C2=O ; CC[n+]1ccccc1CN1C(=O)c2cccc([O-])c2C1=O] | Assay 50 mM PBS solution (THF/water = 1:9, pH 7.4) | Probe 360 nm With Cys 400 nm | Shen, Y.; et al. Spectrochim. Acta Mol. Biomol. Spectrosc. 2017, 185, 371. |
| 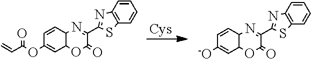 | Assay DMSO: H2O = 4:1 v/v, 10 mM HEPES buffer, pH = 7.4 | Probe 386 nm With Cys 527 nm | Manna, S.; et al. New J. Chem. 2018, 42, 4951. |
| 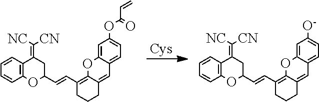 [SMILES: C=CC(=O)Oc1ccc2c(c1)OC1=C(/C=C/C3CC(=C(C#N)C#N)c4ccccc4O3)CCCC1=C2 ; N#CC(C#N)=C1CC(/C=C/C2=C3Oc4cc([O-])ccc4C=C3CCC2)Oc2ccccc21] | PBS buffer (10 mM, pH = 7.4, with 50% of DMSO, v/v) | Probe 550 nm With Cys 600 nm | Qi, Y.; et al. Anal. Chem. 2018, 90, 1014. |

### EXPERIMENTAL PROCEDURES

### Materials and Methods

Reagents and solvents were purchased at the highest commercial quality from Sigma-Aldrich and used without further purification. CHROMASOLV solvents were used as eluents in HPLC. Yields refer to chromatographically (HPLC-MS) and spectroscopically (¹H NMR) homogeneous material, unless otherwise stated. Counter anions are omitted for clarity.

### Analytical HPLC-MS (ESI-MS)

The purity of the compounds was determined with the help of an HPLC-MS apparatus from Waters (Milford, USA) containing the following components: 2695 Separation module, 2696 photodiode array and Waters Micromass ZQ (ESCI ionization mode) detectors. Data acquisition was carried out by MassLynx (V4.1) software.

Column: YMC-Triart C18 3 μM (4.6×150 mm)/Product Nr.TA12S03-1546WT.

Flow: 0.7 mL/min.

Phase A: triethylammonium acetate (TEAAc) buffer (10 mM, pH 7.0) in deionized water.

Phase B: MeCN.

Gradient 80: 5-80B (7 min); 80-80B (2 min); 80-5B (0.5 min); 5-5B (2.5 min).

Gradient 100: 5-100B (7 min); 100-100B (2 min); 100-5B (0.5 min); 5-5B (2.5 min).

### NMR

NMR spectra were recorded on a Bruker Avance (500 and 600 MHz) and an Agilent 400 MR DD2 (400 MHz) instruments and were calibrated using residual non-deuterated solvent as an internal reference.¹ The following abbreviations were used to explain NMR peak multiplicities: s=singlet, d=doublet, t=triplet, q=quartet, m=multiplet, br=broad.

### HRMS

For HRMS (high resolution mass spectra), samples were dissolved in MeCN and analyzed by direct-flow injection (injection volume=5 μL) electrospray ionization time-of-flight (ESI-TOF) mass spectrometry on a Waters Q-ToF Premier instrument in the positive ion mode.

### GENERAL PROCEDURE I

### Preparation of Merocyanine Dyes

A mixture of a respective aldehyde (1 equiv.) and an indolium salt (1 equiv.) in ethanol was refluxed for 1-16 h in the presence of piperidine (0.1-2 equiv.) under Ar. The reaction mixture was allowed to cool slowly to rt, solvent was removed in vacuo and the residue was purified by reversed phase column chromatography (C-18, TEAB buffer (10 mM, pH 7.4)/MeCN or H₂O (0.1% TFA)/MeCN).

### GENERAL PROCEDURE II

### Preparation of Acryloyl Esters

Acryloyl chloride (4-5 equiv.) was added to the mixture of a respective merocyanine dye (1 equiv.) and Et₃N (4-5 eq.) in dry DCM at 0° C. under Ar. After 1 h of stirring at 0° C., the reaction was quenched by adding aqueous NH₄Cl (0.1 M), and organic materials were extracted twice with DCM. The combined extracts were washed with NH₄Cl (0.1 M), dried over Na2SO4, and concentrated in vacuo. The residue was purified by reversed phase column chromatography (C-18, H2O/MeCN).

### GENERAL PROCEDURE III

### Preparation of Acryloyl Esters

Acryloyl chloride (4-5 equiv.) was added to the mixture of a respective merocyanine dye (1 equiv.) and Et₃N (4-5 eq.) in dry DCM at 0° C. under Ar. After 1 h of stirring at 0° C., the reaction was quenched by adding aqueous NH₄Cl (0.1 M), and water-soluble product was extracted twice with H₂O. The combined extracts were washed with DCM, concentrated in vacuo (20 mbar, 20° C.), then purified by reversed phase column chromatography (C-18, H₂O/MeCN).

### SPECTROSCOPIC MATERIALS AND METHODS

Absorption spectra were recorded on a Cary 50 UV-vis spectrometer from Varian. All measurements were performed in 1 cm UV-vis disposable cuvettes (BRAND semi-micro) and air-equilibrated solutions at 25±0.1° C. A total assay volume of 1.0 mL was used for each measurement. UV-vis scan spectra were recorded using following parameters: average time 0.05 s; data interval 1 nm; scan rate 1200 nm/min; with base line correction.

Solutions were prepared in 1.5 mL-vials (Eppendorf® microtubes 3810X) using Vortex Mixers.

Stock solutions of assayed compounds (2-5 mM) were prepared in H₂O-DMSO (1:1), stored at −20° C., and diluted to 1.0 mM with buffer before use. The L-Cys stock solution (20.0 mM) was freshly prepared in buffer before the measurements. HEPES buffer (25 mM, pH 7.4) was used for all measurements.

All aqueous solutions were made up in deionized water with resistivity≥18 MΩ cm⁻¹, obtained using a Millipore purification system (MQ-water).²

### Extinction Coefficients

For a measurement, 1000 μL of buffer and 1-50 μL of probe (1.0 mM) were mixed, and then transferred to a cuvette. Absorbance spectra (250-800 nm) were measured against a blank of the buffer. At least six concentrations of each compound were used to calculate extinction coefficients from the slope of probe concentration vs absorbance plots, using MS Excel software (Microsoft).

In the same manner, ε^{Cys} was determined from the solutions of probe reacted with an excess of Cys (100 μM). The reactions were performed at 37° C. (incubation time 15 min). Blank reactions were run without addition of Cys.

### Evaluation of Stability

For a measurement, 1000 μL of buffer and 16 μL of probe (1.0 mM) were mixed, and then incubated at +4° C. and at +37° C. for 5 h. The resulting mixtures were transferred to a cuvette, and the absorbance (250-800 nm) was measured. Each measurement was done in triplicate.

### Evaluation of Solubility

In the experiments, 5-10 mg of dried material was suspended in 250-500 μL of H2O at RT. The resulting suspension was centrifuged for 10 min at RT (16000 rcf). UV-vis of supernatant were recorded in buffer (25 mM HEPES pH 7.4) at RT. Each measurement was done in triplicate. The pellet was dried in vacuo for 16 h, and then weighed.

As mentioned above, the final products were obtained in a two-step synthesis (condensation and acrylation; Schemes 1 to 3). Overall yields 21-59%, except for 6% in case of MF65. The products were characterized by HPLC-MS, ¹H and ¹³C NMR, and UV-Vis.

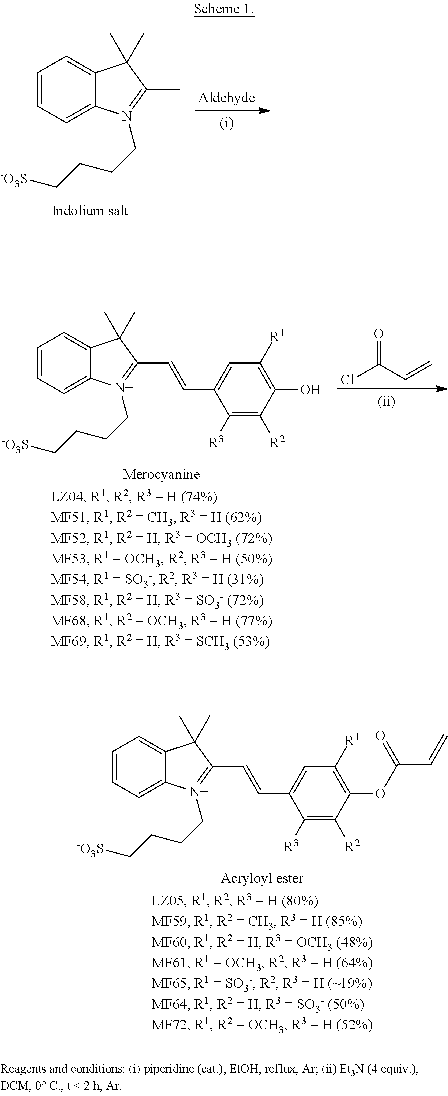

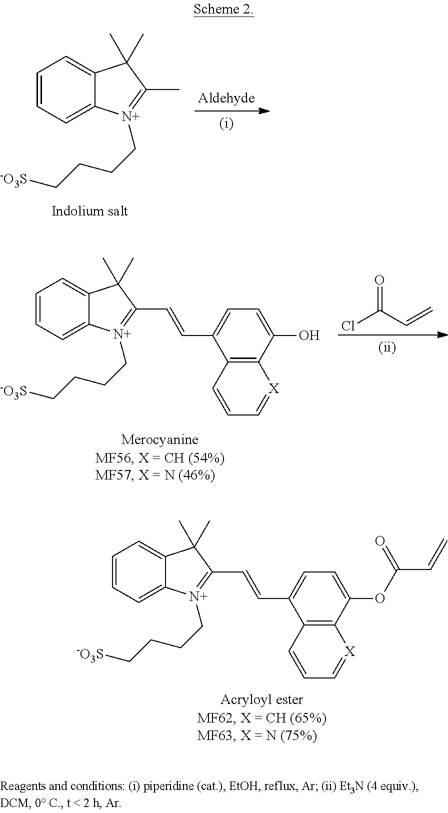

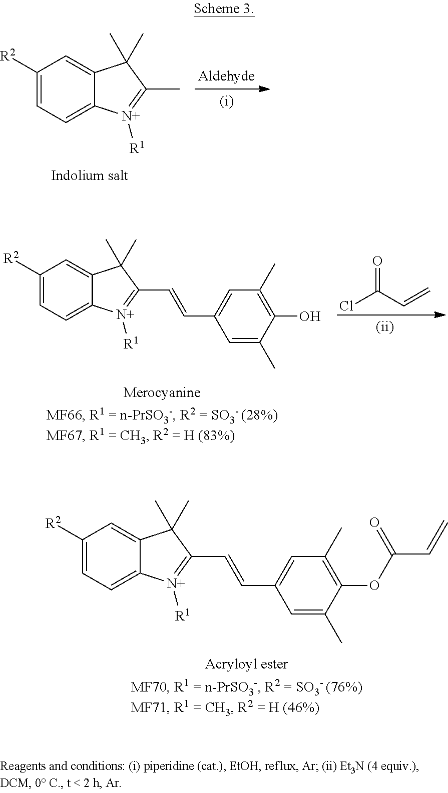

### UV-Vis and Cys Response

UV-vis of dyes as obtained was evaluated. Selected substituents on the benzene ring helped to increase the value for the initial merocyanine dye. Most remarkable results were observed for the preferred intermediate compounds MF56, MF57 and MF66.

TABLE 1
Spectral properties of dyes according to the present invention.*

| Entry | λₘₐₓ (ε mM⁻¹cm⁻¹) nm |
|---|---|
| LZ04 | 527 (36.6) |
| (contr.) | 527 (45.6)** |
| LZ06 | 520 (27-37) I⁻ not quant. |
| (contr.) | 520 (37-53) I⁻ not quant. |
| MF51 | 550 (73.9) |
| MF52 | 529 (70.1) |
| MF53 | 548 (65.3) |
| MF54 | 528 (44.9) |
| MF56 | 582 (109.7) |
| MF57 | 572 (106.3) |
| MF58 | 514 (48.1, broad) |
| MF66 | 556 (122.6) |
| | 556 (138.6)** |
| MF67 | 546 (67.4) |
| | 546 (84.9)** |
| MF68 | 575 (70.0) |

*UV-vis spectra were recorded in aqueous buffer (25 mM HEPES pH 7.4). The concentration of the probes was 1-24 μM. At least six concentrations of each compound were used in the experiments.
**The experiments were performed in the same buffer at pH 8.0.

Spectra recorded in the presence of Cys confirmed a colorimetric response through the cleavage of acryloyl ester. Probes as synthesized showed significant bathochromic shifts to the green and yellow range of the visible spectrum (Table 2). Moreover, their spectral profiles were advantageously fulfilling the wavelength requirement for the Cys sensing application in Cedex system (340, 378, 409, 480, 512, 520, 552, 583, 629, 652, 659 and 800 nm).

TABLE 2
Spectral properties and Cys-response screening of
compounds according to the present invention.*

| | λₘₐₓ | λ^{Cys} | |
|---|---|---|---|
| | (ε mM⁻¹cm⁻¹) | (ε^{Cys} mM⁻¹cm⁻¹) | |
| Entry | nm | nm | SNR |
| LZ05 (contr.) | 527 (1.3), 389 (17.3) | 527 (31.9) | 12 |
| LZ07 (contr.) | 520 (0.6), 383 (16.9) | 520 (31.5) | 16 |
| MF59 | 550 (0.7), 391 (16.7) | 550 (17.9) | 20 |
| | | 550 (52.9)** | 44** |
| MF60 | 530 (2.1), 409 (14.3) | 529 (67.8) | 14 |
| MF61 | 548 (0.4), 397 (20.4) | 549 (61.7) | 36 |
| MF62 | 582 (1.5), 419 (8.5), | 582 (105.3) | 20 |
| | 287 (7.7) | | |
| MF63 | 572 (2.9), 397 (14.0) | 572 (93.4) | 10 |
| MF64 | 514 (0.7), 350 (8.0) | 514 (32.1) | 14 |
| MF65 | 528 (1.2), 381 (17.7) | 528 (44.3) | 16 |
| MF70 | 400 (21.9) | 556 (23.6) | 118 |
| | 400 (16.6)* | 556 (55.8)** | 140** |
| MF71 | 546 (0.6), 387 (29.1) | 547 (33.7) | 48 |
| | 546 (0.8), 385 (25.7)* | 547 (69.6)** | 53** |
| MF72 | 396 (14.2) | 575 (31.0) | 103 |

*UV-vis spectra were recorded before and after addition of an excess of Cys (100 μM) in aqueous buffer (25 mM HEPES pH 7.4). The concentration of the probes was 1-24 μM. The reactions with Cys were performed at 37° C. (incubation time 15 min). SNR was estimated from the values obtained after reaction with Cys (ε^{Cys}) and of blank measurements (ε^{blank}). At least six concentrations of each compound were used in the experiments.
**The experiments were performed in the same buffer at pH 8.0.

### Stability

The stability is another important parameter for the evaluation of colorimetric probes for biological assays. The probes for commercial applications have to be storable at 4° C. for several months. In addition, the stability has to be examined under assay conditions of Cedex Bio HT analyzer (37° C.).

The inventors qualitatively examined the stability of probes in buffer (10 mM HEPES pH 7.4) at 4° C. and 37° C. in order to simulate usual conditions of storage and assays. Solutions of each probe were monitored by UV-vis for 5 h. The results of all spectroscopic evaluations are summarized in Table 3.

TABLE 3
* Stability of compounds of the present invention

| Entry | Hydrolysis @4° C., % | Hydrolysis @37° C., % |
|---|---|---|
| LZ05 (contr.) | 8.4 ± 0.2 | 36.9 ± 0.4 |
| LZ07 (contr.) | 7.3 ± 0.1** | 30.1 ± 0.2** |
| MF59 | 1.2 ± 0.0 | 4.7 ± 0.1 |
| MF60 | 9.8 ± 0.3 | 41.8 ± 0.9 |
| MF61 | 4.8 ± 0.1 | 29.1 ± 1.9 |
| MF62 | 7.6 ± 0.5 | 40.4 ± 0.6 |
| MF63 | 9.8 ± 1.5 | 55.3 ± 0.7 |
| MF64 | 8.9 ± 0.2 | 45.2 ± 1.4 |
| MF65 | 7.2 ± 0.2 | 28.9 ± 1.4 |
| MF70 | 0.2 ± 0.0 | 0.8 ± 0.0 |
| MF71 | 1.0 ± 0.1 | 2.3 ± 0.2 |
| MF72 | 0.8 ± 0.1 | 2.8 ± 0.1 |

* The experiments were performed in aqueous buffer (25 mM HEPES pH 7.4) at 4° C. and 37° C., with a concentration of 15.6 μM (incubation time 5 h). The amount of hydrolyzed probe was determined using ε values derived from merocyanine analogues (Table 1). Each measurement was done in triplicate.
**The amount of hydrolyzed probe was determined using ε value derived from Cys response screening (Table 2).

### Solubility

The aqueous solubility of probes is another important property for the performance of compounds in biological assays. The inventors determined the solubility in water of the probes according to the invention using UV-vis. First, oversaturated mixtures of each probe were prepared. The mixtures were centrifuged, and then the concentrations in probes of supernatant were examined by UV-vis. To further validate the results, the concentrations were calculated from the isolated pellet weight. As seen in Table 4, the results as determined with both approaches are in the same range, and followed the same trend. Preferred probe MF70, containing two sulpho-groups, is highly water-soluble in comparison to its analogues LZ05 and MF59 containing only one sulpho-group. Also, the solubility of MF70 is superior to the compound LZ07 known from literature.

TABLE 4
* Solubility.

| Entry | Solubility (by UV-vis), mM | Solubility (by weight), mM |
|---|---|---|
| LZ05 (contr.) | 11.0 ± 1.0 | 12 |
| LZ07 (contr.) | 15.4 ± 1.5 | 17 |
| MF59 | 5.8 ± 0.2 | 2 |
| MF70 | ≥41 | ≥47 |

*In the experiments 5-10 mg of dried material was suspended in 250-500 μL of H₂O at RT. The UV-vis of supernatants was recorded in buffer (25 mM HEPES pH 7.4) at RT. Each measurement was done in triplicate. Probes were used as obtained from synthesis.

### Probe MF70 Performance in Cedex Bio HT

The rapid kinetic profile, stability and low background signal as obtained were encouraging to further use MF70 for Cys-sensing in Cedex Bio HT. Reagent solution was treated with various Cys-concentrations (0.5-7.6 mM) in a feed medium, which is used for monoclonal antibody production. As shown in FIG. 3, progressively enhanced absorbance was observed with the increasing amount of Cys. Under these conditions, a reliable response was obtained over the period of one week (Table 5), with corresponding detection limit of 3.6 μM Cys, and limit of blank 2.2 μM Cys.

TABLE 1
Spike-recovery in a feed medium (DMT118F.01 with Cys).

| Spiked Cys, mM | n (samples) | Found, mM | CV, % | Recovery, % |
|---|---|---|---|---|
| 0.5 | 21 | 0.64 | 1 | 96 |
| 1.5 | 21 | 1.64 | 1 | 99 |
| 3.0 | 21 | 3.03 | 1 | 96 |
| 4.5 | 21 | 4.38 | 1 | 94 |
| 6.0 | 21 | 5.83 | 1 | 94 |

In conclusion, the probes according to the present invention, and preferably probe MF70 meet the requirements for commercial assays. The merocyanine dye scaffold ensures bright chromogenic signal. Methyl groups in the ortho-position and sulfonic acid groups seem to secure stability against hydrolysis and aqueous solubility, respectively.

1. A compound according to Formula I 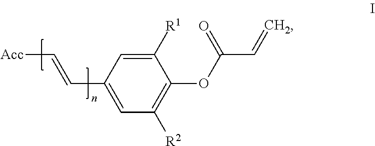
wherein
R¹ and R² are independently selected from R³, O—R³, S—R³, SO₃⁻, SO₃—R³, wherein R³ is selected from C₁-C₁₈ alkyl, and a polyethylene glycol (PEG) residue,
Acc is selected from the group selected from Formula II 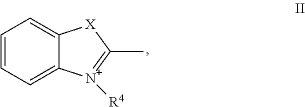
wherein X is selected from —N(CH₃)—, —S—, —Se—, —O—, and —C(CH₃)₂—, 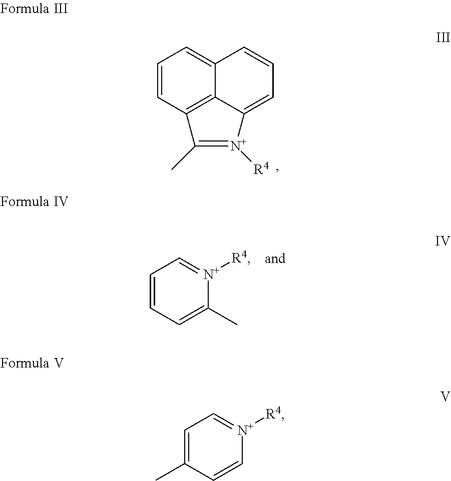
wherein optionally in each of Formula II to V an aromatic ring is substituted with 1, 2 or 3 SO₃⁻ groups,
R⁴ is selected from the group of C₁-C₁₈ alkyl, C₁-C₆ cycloalkyl, and (CH₂)ₘ—SO₃⁻, wherein m is an integer selected from 1 to 18, and
n is selected from 1, 2 and 3,
and suitable salts and solvates thereof.

2. The compound of Formula I according to claim 1, wherein R¹ and R² are independently selected from R³, O—R³, S—R³, SO₃⁻, SO₃—R³, wherein R³ is selected from C₁-C₆ alkyl, and a polyethylene glycol (PEG) residue,
Acc is Formula II 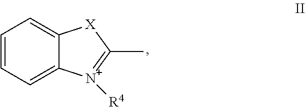
wherein X is selected from —N(CH₃)—, —S—, —Se—, —O—, and —C(CH₃)₂—, optionally the aromatic ring is substituted with 1, 2 or 3 SO₃⁻ groups, R⁴ is selected from C₁-C₆ alkyl, C₁-C₆ cycloalkyl, and (CH₂)ₘ—SO₃⁻, wherein m is an integer from 1 to 6, and n is 1, and suitable salts and solvates thereof.

3. The compound of Formula I according to claim 1, wherein R¹ and R² are independently selected from R³, O—R³, S—R³, SO₃⁻, SO₃—R³, wherein R³ is selected from C₁-C₃ alkyl, and a polyethylene glycol (PEG) residue,
Acc is Formula II 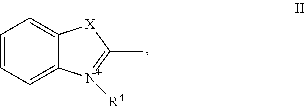
wherein X is —C(CH₃)₂—, optionally the aromatic ring is substituted with 1, 2 or 3 SO₃⁻ groups, R⁴ is (CH₂)ₘ—SO₃⁻, wherein m is an integer from 1 to 6, and n is 1, and suitable salts and solvates thereof.

4. The compound of Formula I according to the following formulae VI to IX 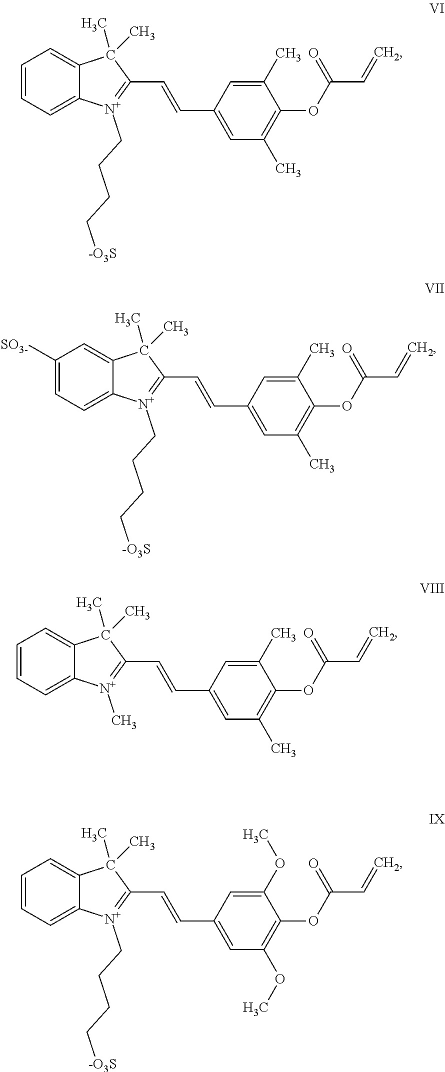
and suitable salts and solvates thereof.

5. A method for preparing a compound according to Formula I according to claim 1, comprising the steps of:
a) suitably reacting a compound of Formula VI 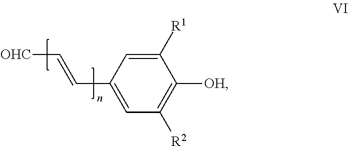
wherein R¹ and R² are as defined in claim 1, and n is 1 or 2, with a compound of Formula II 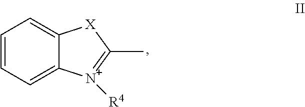
or with a compound of formula III 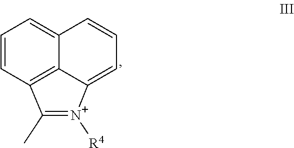
or with a compound of Formula IV 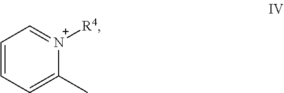
or with a compound of Formula V 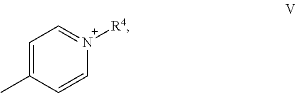
wherein in each of Formulae II to V optionally an aromatic ring is substituted with 1, 2 or 3 SO₃⁻ groups, R⁴ is selected from C₁-C₁₈ alkyl, C₁-C₆ cycloalkyl, and (CH₂)ₘ—SO₃⁻, and wherein m is an integer from 1 to 18, to obtain a compound of Formula VIII 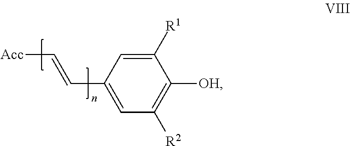
wherein R¹, R² and Acc are as defined in claim 1, and n is 1 or 2, and
b) suitably reacting the compound of formula VIII with acryloyl chloride.

6. A method for detecting cysteine in a test sample, comprising the following steps of:
a) measuring of UV/Vis absorbance of a solution of a compound as defined in claim 1 in a suitable solvent before and after being contacted with a prospectively cysteine-containing test sample, and
b) determining the difference in absorbance by comparison of the UV/Vis spectra as measured in step a), and
c) detecting cysteine in said test sample based on said difference in absorbance as determined in step b).

7. The method according to claim 5, wherein the UV/Vis absorbance is measured at discrete wavelengths in the range of from 200 nm to 1000 nm.

8. The method according to claim 7, wherein the wavelengths are selected from the group consisting of 340, 378, 409, 480, 512, 520, 552, 583, 629, 659 and 800 nm.

9. The method according to claim 6, wherein the solvent is an aqueous solvent.

10. The method according to claim 6, wherein said determining the difference in absorbance is by a visual inspection of a color change.

11. The method according to claim 6, wherein said determining the difference in absorbance is by Cedex

12. A kit for detecting cysteine in a test sample, comprising a vial or container comprising a predetermined quantity of a compound according to claim 1, together with a manual for using said kit.

13. (canceled)
